(19) United States
(12) Patent Application Publication
(10) Pub. No.: US 2026/0294764 A1
(43) Pub. Date: Oct. 1, 2026

(54) ARTIFICIAL NAIL COMPOSITION AND ARTIFICIAL NAIL

(71) Applicant: SHOFU INC., Kyoto-shi (JP)

(72) Inventors: Yuko NAKASHOJI, Kyoto-shi (JP); Shun SHIMOSOYAMA, Kyoto-shi (JP)

(73) Assignee: SHOFU INC., Kyoto-shi (JP)

(21) Appl. No.: 19/632,708

(22) Filed: Mar. 30, 2026

(30) Foreign Application Priority Data
Mar. 31, 2025 (JP) 2025-057456
Feb. 26, 2026 (JP) 2026-030078

(51) Int. Cl. A61K 8/44; A61K 8/02; A61K 8/37; A61K 8/49; A61K 8/55; A61K 8/73; A61Q 3/02

(52) U.S. Cl. CPC A61K 8/44; A61K 8/02; A61K 8/37; A61K 8/49; A61K 8/4973; A61K 8/55; A61K 8/731; A61Q 3/02; A61K 2800/10; A61K 2800/412; A61K 2800/652; A61K 2800/95

(57) ABSTRACT

An object of the present invention is to provide an artificial nail composition for forming an artificial nail having mattness with a subdued gloss and a smooth texture. The artificial nail composition of the present invention includes: a component (A) that is a polymerizable compound; a component (B) that is cellulose particles; and a component (C) that is a polymerization initiator.

### FIELD OF INVENTION

The present invention relates to an artificial nail composition and an artificial nail.

### BACKGROUND

Among artificial nails such as nail polish, gel nails, and acrylic nails, the gel nails are widely used from the viewpoint of good operability, long-term maintainability on nails or artificial nails, and low odorousness. There are demands for, as one of gel nail designs, gel nails having mattness with a subdued gloss (without sheen), and one of techniques of realizing such gel nails having mattness includes a technique of subduing a gloss by adding a filler to a photocurable artificial nail composition to form an uneven cured film surface so that diffuse reflection of light is caused.

JP 7014970B describes that a gloss-subdued artificial nail composition is obtained by containing a compound having a (meth)acryloyl group, a photoinitiator, and (meth)acrylic polymer particles.

### SUMMARY OF INVENTION

The artificial nail composition of JP 7014970B has mattness with a subdued gloss, but has a problem such as deterioration in smoothness of a cured coating surface. As described above, it is difficult for the conventional artificial nail composition to achieve both the mattness and a smooth texture.

An object of the present invention is to provide an artificial nail composition for forming an artificial nail having mattness with a subdued gloss and a smooth texture.

The above-described problem is solved by the following present invention. That is, an artificial nail composition of the present invention includes: a component (A) that is a polymerizable compound; a component (B) that is cellulose particles; and a component (C) that is a polymerization initiator.

As one embodiment of the artificial nail composition of the present invention, preferably, the component (B) that is cellulose particles has an average particle size of 3 to 30 μm.

As another embodiment of the artificial nail composition of the present invention, a content of the component (B) that is cellulose particles is 5 to 60 mass % with respect to a total amount of the artificial nail composition.

As still another embodiment of the artificial nail composition of the present invention, the component (A) that is a polymerizable compound is at least one selected from the group consisting of a urethane (meth)acrylate oligomer and a polymerizable monomer. In addition, preferably, the urethane (meth)acrylate oligomer is at least one selected from the group consisting of a urethane mono(meth)acrylate oligomer, a urethane di(meth)acrylate oligomer, and a urethane hexa(meth)acrylate oligomer. In addition, preferably, the polymerizable monomer is at least one selected from the group consisting of isobornyl (meth)acrylate, tetrahydrofurfuryl (meth)acrylate, hydroxypropyl (meth)acrylate, triethylene glycol di(meth)acrylate, (meth)acryloylmorpholine, trimethylolpropane tri(meth)acrylate, ethoxyethylene glycol (meth)acrylate, and isoprenyl (meth)acrylate.

As still another embodiment of the artificial nail composition of the present invention, preferably, the component (C) that is a polymerization initiator is a photopolymerization initiator.

As still another embodiment of the artificial nail composition of the present invention, preferably, the artificial nail composition includes 39 to 94 mass % of the component (A): a polymerizable compound, 5 to 60 mass % of the component (B): cellulose particles; and 0.1 to 10 mass % of the component (C): polymerization initiator.

An artificial nail of the present invention is obtained from the artificial nail composition. As an embodiment of the artificial nail of the present invention, preferably, a surface of the artificial nail has arithmetic average roughness (Ra) of 0.2 to 1.3 μm.

According to the present invention, it is possible to provide the artificial nail composition for forming an artificial nail having mattness with a subdued gloss and a smooth texture.

### DETAILED DESCRIPTION OF THE PREFERRED EMBODIMENTS

An artificial nail composition of the present invention contains: a component (A) that is a polymerizable compound; a component (B) that is cellulose particles; and a component (C) that is a polymerization initiator.

### [Polymerizable Compound]

A component (A) that is a polymerizable compound is a substance that reacts by external energy such as light or heat to form a polymer. The component (A) imparts properties such as curability, surface hardness, strength, flexibility, durability, and workability to the artificial nail composition of the present invention. The component (A) is not particularly limited as long as the component (A) is a compound (for example, a polymerizable monomer, an oligomer, a polymer, or the like) having an ethylenically unsaturated group as a polymerizable functional group, and a known polymerizable compound can be used. Specific examples of the ethylenically unsaturated group include a (meth)acryloyl group, a (meth)acryloyloxy group, a (meth)acrylamide group, an isoprenyl group, a vinyl group, a vinyl ether group, a methyl vinyl ether group, an allyl group, an allyl ether group, and a maleimide group and are not limited thereto. Among the ethylenically unsaturated groups, from the viewpoint of curability, surface hardness, strength, flexibility, and durability, at least one selected from the group consisting of a (meth)acryloyl group, a (meth)acryloyloxy group, and an isoprenyl group is preferable.

Note that, in this specification, the term, (meth)acryloyl, encompasses both acryloyl and methacryloyl, the term, (meth)acrylate, encompasses both acrylate and methacrylate, the term, (meth)acryloyloxy, encompasses both acryloyloxy and methacryloyloxy, and the term, (meth)acrylamide, encompasses both acrylamide and methacrylamide.

Specific examples of the component (A) having one ethylenically unsaturated group in one molecule include polymerizable monomers such as methacrylic acid, acrylic acid, urethane mono(meth)acrylate, methoxyethylene glycol (meth)acrylate, methoxypolyethylene glycol (meth)acrylate, ethoxyethylene glycol (meth)acrylate, ethoxypolyethylene glycol (meth)acrylate, phenoxyethylene glycol (meth)acrylate, phenoxypolyethylene glycol (meth)acrylate, 2-hydroxy-3-phenoxypropyl (meth)acrylate, 2-(meth)acryloyloxyethyl succinate, 2-(meth)acryloyloxyethyl phthalate, 2-(meth)acryloyloxypropyl hexaphthalate, stearyl (meth)acrylate, methyl (meth)acrylate, ethyl (meth)acrylate, hydroxyethyl (meth)acrylate, hydroxypropyl (meth)acrylate, hydroxy butyl (meth)acrylate, 3-chloro-2-hydroxypropyl (meth)acrylate, isobornyl (meth)acrylate, tetrahydrofurfuryl (meth)acrylate, 2-(meth)acryloyloxyethyl dihydrogen phosphate, 2-(meth)acryloyloxyethyl phenyl hydrogen phosphate, 10-(meth)acryloyloxydecyl dihydrogen phosphate, 6-(meth)acryloyloxyhexyl dihydrogen phosphate, 2-(meth)acryloyloxyethyl-2-bromoethyl hydrogen phosphate, methylol (meth)acrylamide, dimethyl (meth)acrylamide, (meth)acryloylmorpholine, and vinyl chloride, an oligomer of the polymerizable monomers, and a polymer of the polymerizable monomers, and are not limited thereto. The oligomer and the polymer may contain one or two or more of the examples of polymerizable monomers. These may be used singly or in combination of two or more kinds thereof.

Note that the term, oligomer, in this specification means a polymer obtained by polymerizing two to several tens of polymerizable monomers. In addition, the term, polymer, means a polymer obtained by polymerizing several tens or more polymerizable monomers and means a polymer other than oligomers.

Specific examples of the component (A) having two ethylenically unsaturated groups in one molecule include polymerizable monomers such as urethane di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octanediol di(meth)acrylate, glycerin di(meth)acrylate, ethylene glycol di(meth)acrylate, triethylene glycol di(meth)acrylate, polyethylene glycol di(meth)acrylate, propylene glycol di(meth)acrylate, polypropylene glycol di(meth)acrylate, ethoxylated polypropylene glycol di(meth)acrylate, ethoxylated propylene glycol di(meth)acrylate, ethoxylated bisphenol A di(meth)acrylate, propoxylated bisphenol A di(meth)acrylate, propoxylated ethoxylated bisphenol A di(meth)acrylate, tricyclodecane dimethanol di(meth)acrylate, isoprenyl (meth)acrylate, and bis[2-(methacryloyloxy)ethyl]phosphate; an oligomer of the polymerizable monomers; and a polymer of the polymerizable monomers and are not limited thereto. The oligomer and the polymer may contain one or two or more of the examples of polymerizable monomers. These may be used singly or in combination of two or more kinds thereof.

Specific examples of the component (A) having three or more ethylenically unsaturated groups in one molecule include polymerizable monomers such as urethane tri(meth)acrylate, urethane tetra(meth)acrylate, urethane penta(meth)acrylate, urethane hexa(meth)acrylate, trimethylolpropane tri(meth)acrylate, ethoxylated trimethylolpropane tri(meth)acrylate, ethoxylated glycerin tri(meth)acrylate, ditrimethylolpropane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, ethoxylated isocyanuric acid tri(meth)acrylate, tetramethylolmethane tetra(meth)acrylate, dipentaerythritol tetra(meth)acrylate, ditrimethylolpropane tetra(meth)acrylate, dipentaerythritol penta(meth)acrylate, caprolactone-modified pentaerythritol penta(meth)acrylate, dipentaerythritol hexa(meth)acrylate, and caprolactone-modified pentaerythritol hexa(meth)acrylate; an oligomer of the polymerizable monomers; and a polymer of the polymerizable monomers and are not limited thereto. The oligomer and the polymer may contain one or two or more of the examples of polymerizable monomers. These may be used singly or in combination of two or more kinds thereof.

A molecular weight (Mw) of a polymerizable monomer of the component (A) is not particularly limited and is preferably less than 1,000. A weight-average molecular weight (Mw) of the oligomer of the component (A) is not particularly limited and is, for example, 30,000 or less, preferably 200 to 30,000, more preferably 300 to 30,000, and still more preferably 700 to 10,000. A weight-average molecular weight (Mw) of the polymer of the component (A) is not particularly limited and is, for example, more than 30,000, preferably more than 30,000 and 100,000 or less. Within this range, properties such as the curability, the surface hardness, the strength, the flexibility, and the durability can be improved. Note that, in this specification, as the weight-average molecular weight (Mw), a value measured by gel permeation chromatography (GPC) using polystyrene as a standard substance is used.

The polymerizable monomer is a liquid under a 23° C. atmosphere and preferably has fluidity. Specifically, a viscosity at 23° C. and at a shear rate of 10 s⁻¹ measured using a rheometer which is a dynamic viscoelasticity measuring device is preferably 20,000 mPa·s or less, more preferably 10,000 mPa·s or less, still more preferably 5,000 mPa·s or less, and particularly preferably 1,000 mPa·s or less. The oligomer may or may not have fluidity under the 23° C. atmosphere. Specifically, the viscosity at 23° C. and at the shear rate of 10 s⁻¹ measured using the rheometer which is the dynamic viscoelasticity measuring device is preferably 7,000 mPa·s or more, more preferably 7,000 to 3,000,000 mPa·s, still more preferably 7,000 to 2,500,000 mPa·s, and particularly preferably 7,000 to 2,200,000 mPa·s. The polymer may or may not have fluidity under the 23° C. atmosphere.

Two or more kinds of the components (A) can be selected to make the artificial nail composition. Among these examples, it is preferable to use at least one selected from the group consisting of methacrylic acid, acrylic acid, urethane mono(meth)acrylate, urethane di(meth)acrylate, urethane tri(meth)acrylate, urethane tetra(meth)acrylate, urethane penta(meth)acrylate, urethane hexa(meth)acrylate, isobornyl (meth)acrylate, tetrahydrofurfuryl (meth)acrylate, hydroxypropyl (meth)acrylate, triethylene glycol di(meth)acrylate, (meth)acryloylmorpholine, bis[2-(methacryloyloxy)ethyl]phosphate, trimethylolpropane tri(meth)acrylate, ethoxyethylene glycol (meth)acrylate, isoprenyl (meth)acrylate, an oligomer thereof, and a polymer thereof. The oligomer and the polymer may contain one or two or more of the examples of polymerizable monomers as a constituent unit. In addition, the component (A) more preferably contains the oligomer and the polymerizable monomer, further preferably contains at least one urethane (meth)acrylate oligomer selected from the group consisting of a urethane mono(meth)acrylate oligomer, a urethane di(meth)acrylate oligomer, and a urethane hexa(meth)acrylate oligomer, and at least one polymerizable monomer selected from the group consisting of isobornyl (meth)acrylate, tetrahydrofurfuryl (meth)acrylate, hydroxypropyl (meth)acrylate, triethylene glycol di(meth)acrylate, (meth)acryloylmorpholine, trimethylolpropane tri(meth)acrylate, ethoxyethylene glycol (meth)acrylate, and isoprenyl (meth)acrylate, and particularly preferably contains a urethane di(meth)acrylate oligomer and at least one polymerizable monomer selected from the group consisting of isobornyl (meth)acrylate and triethylene glycol di(meth)acrylate. When the component (A) contains the oligomer and the polymerizable monomer, properties such as the curability, the surface hardness, the strength, the flexibility, the durability, and the workability can be improved.

A content of the component (A) is not particularly limited, and the content of the component (A) is, with respect to the total amount of the composition, preferably within a range of 39 to 94 mass %, more preferably within a range of 49 to 89 mass %, and still more preferably within a range of 49 to 79 mass %. Within the ranges, the properties such as mattness, a texture, adhesion resistance, an antifouling property, transparency, the surface hardness, the strength, the flexibility, the durability, and the workability can be improved.

### [Cellulose Particles]

A component (B) that is cellulose particles imparts mattness with a subdued gloss and a smooth texture to the artificial nail composition of the present invention. Further, the component (B) improves adhesion resistance and an antifouling property and imparts properties such as transparency, strength, flexibility, durability, and workability to the artificial nail composition.

The component (B) is particles including, as a main component, cellulose or cellulose acetate and can be used without any limitation as long as the component (B) is not dissolved in another component. In addition, a shape of the component (B) is not particularly limited, and any particle shape such as a spherical shape, a needle shape, a plate shape, a crushed shape, or a scaly shape can be used without any limitation. Among the shapes, the spherical shape is preferable.

A structure of the component (B) is not particularly limited, and any structure such as a porous structure, a non-porous structure, a solid structure, a hollow structure, or a core-shell structure can be used. Among the structures, the non-porous structure is preferable.

An average particle size of the component (B) is not particularly limited and is preferably within a range of 3 to 30 μm, more preferably within a range of 3 to 20 μm, and still more preferably within a range of 5 to 10 μm. Within the ranges, the mattness, the texture, the adhesion resistance, the antifouling property, the transparency, and the durability can be improved.

Note that the term, the average particle size, in this specification means a value of an integrated volume 50% particle size obtained by measuring a particle grain-size distribution with a laser diffraction particle grain-size distribution measuring machine.

A content of the component (B) is not particularly limited and is, with respect to the total amount of the composition, preferably within a range of 5 to 60 mass %, more preferably within a range of 10 to 50 mass %, and still more preferably within a range of 20 to 35 mass %. Within the ranges, the mattness, the texture, the adhesion resistance, the antifouling property, the transparency, the strength, the flexibility, the durability, and the workability can be improved.

### [Polymerization Initiator]

The component (C) that is a polymerization initiator is a compound for initiating a polymerization reaction of the component (A). A kind of component (C) is not particularly limited, and a known polymerization initiator can be used. Examples of the component (C) include a photopolymerization initiator that serves as a starting point of a polymerization reaction by irradiation with an energy ray such as a visible light ray, an ultraviolet ray, an X-ray, or an electron beam, a thermal polymerization initiator that serves as a starting point of a polymerization reaction by heating to a certain temperature or higher, and a chemical polymerization initiator of a type in which two or more agents that serve as starting points of a polymerization reaction are mixed by mixing with a specific substance, and it is preferable to use the photopolymerization initiator. Examples of the photopolymerization initiator include a radical polymerization initiator that generates a radical by irradiation with an energy ray, a cationic polymerization initiator that generates a cation, an anionic polymerization initiator that generates an anion, and it is preferable to use the radical polymerization initiator. Specific examples of the radical photopolymerization initiator include benzoin ethers, benzyl ketals, α-dialkoxyacetophenones, α-hydroxyalkylphenones, α-aminoalkylphenones, acylphosphine oxides, benzophenones, thioxanthones, and titanocenes and are not limited thereto. In addition, two or more of these polymerization initiators can be selected to make the artificial nail composition. Among the examples, it is preferable to use at least one selected from the group consisting of α-hydroxyalkylphenones and acylphosphine oxides.

Specific examples of the α-hydroxyalkylphenones include 1-hydroxycyclohexyl phenyl ketone, 2-hydroxy-2-methyl-1-phenyl-propan-1-one, and α-aminoalkylphenone. Specific examples of the acylphosphine oxides include 2,4,6-trimethylbenzoyldiphenylphosphine oxide, 2,4,6-trimethylbenzoylphenylethoxyphosphine oxide, and bis(2,4,6-trimethylbenzoyl)phenylphosphine oxide. Among the examples, it is preferable to use at least one selected from 2,4,6-trimethylbenzoyldiphenylphosphine oxide, 2,4,6-trimethylbenzoylphenylethoxyphosphine oxide, and bis(2,4,6-trimethylbenzoyl)phenylphosphine oxide.

A content of the component (C) is not particularly limited and is, with respect to the total amount of the composition, preferably within a range of 0.1 to 10 mass %, more preferably within a range of 0.1 to 6 mass %, and still more preferably within the range of 0.5 to 6 mass %. Within the ranges, properties such as the curability, the surface hardness, the strength, the flexibility, and the durability can be improved.

### [Other Components]

The artificial nail composition of the present invention can contain components other than the components (A) to (C) as long as the effects of the present invention are not impaired. Examples of the other components include a supplement agent, an additive, a colorant, a leveling agent, a plasticizer, an antioxidant, a polymerization accelerator, a polymerization inhibitor, a coagulant, a preservative, a wax, a thickener, a fragrance, a UV blocking agent, a diffuser, a defoamer, a dispersant, a filler, a surfactant, a pigment, a dye, an excipient, an ion-releasing agent, an antibacterial agent, a chain transfer agent, and a silane coupling agent, which are widely used in artificial nail compositions. These examples may be used singly or in combination of two or more kinds thereof.

As the polymerization accelerator, a polyfunctional thiol compound having two or more thiol groups in one molecule can be used. By using the polyfunctional thiol compound, a curing reaction proceeds without being inhibited by oxygen. Examples of specific compounds as the polyfunctional thiol compounds include 1,2-ethanedithiol, 1,2-propanedithiol, 1,3-propanedithiol, 1,3-butanedithiol, 2,3-butanedithiol, 1,5-pentanedithiol, 1,6-hexanedithiol, 1,8-octanedithiol, 1,9-nonanedithiol, 1,10 decanedithiol, 1,2-benzenedithiol, 1,3-benzenedithiol, 1,4-benzenedithiol, 3,6-dichloro-1,2-benzenedithiol, toluene-3,4-dithiol, 1,5-naphthalenedithiol, ethylene glycol bis(thioglycolate), ethylene glycol bis(3-mercaptopropionate), 1,4-butanediol bisthioglycolate, tetraethylene glycol bis(3-mercaptopropionate), trimethylolpropane tris(thioglycolate), trimethylolpropane tris(3-mercaptopropionate), trimethylolpropane tris(3-mercaptobutyrate), tris[(3-mercaptopropionyloxy)-ethyl]isocyanurate, pentaerythritol tetrakis(thioglycolate), pentaerythritol tetrakis(3-mercaptopropionate), dipentaerythritol hexakis (3-mercaptopropionate), 1,4-bis(3-mercaptobutyryloxy)butane, pentaerythritol tetrakis(3-mercaptobutyrate), 1,3,5-tris(3-mercaptobutyloxyethyl)-1,3,5-triazine-2,4,6 (1H,3H,5H)-trione, dimercaptodiethylsulfide, 1,8-dimercapto-3,6-dithiaoctane, 1,2-bis[(2-mercaptoethyl)thio]-3-mercaptopropane, tetrakis(7-mercapto-2,5-dithiaheptyl)methane, trithiocyanuric acid, 1,2-benzenedimethane, thiol, 4,4′-thiobisbenzenethiol, 2-di-n-butylamino-4,6-dimercapto-s-triazine, 2,5-dimercapto-1,3,4-thiadiazole, 1,8-dimercapto-3,6-dioxaoctane, 1,5-dimercapto-3-thiapentane, tris(2-hydroxyethyl)isocyanurate trimercaptopropionate, 1,4-dimethylmercaptobenzene, 2,4,6-trimercapto-s-triazine, 2-(N,N-dibutylamino)-4,6-dimercapto-s-triazine, bis(4-(2-mercaptopropoxy)phenyl)methane, 1,1-bis(4-(2-mercaptopropoxy)phenyl)ethane, 2,2-bis(4-(2-mercaptopropoxy)phenyl)propane, 2,2-bis(4-(2-mercaptopropoxy)phenyl)butane, 1,1-bis(4-(2-mercaptopropoxy)phenyl)isobutane, 2,2-bis(4-(2-mercaptopropoxy)-3-methylphenyl)propane, 2,2-bis(4-(2-mercaptopropoxy)-5-methylphenyl)propane, bis(2-(2-mercaptopropoxy)-5-methylphenyl)methane, 2,2-bis(4-(2-mercaptopropoxy)-3-t-butylphenyl)propane, tris(4-(2-mercaptopropoxy)phenyl)methane, 1,1,1-tris(4-(2-mercaptopropoxy)phenyl)ethane, bis(4-(2-mercaptobutoxy)phenyl)methane, 2,2-bis(4-(2-mercaptobutoxy)phenyl)propane, tris(4-(2-mercaptobutoxy)phenyl)methane, 1,3,5-triazine-2,4,6-trithiol, and an alkyl vinyl ether adduct thereof. Here, the examples thereof are not limited thereto. These polymerization accelerators may be used singly or in combination of two or more kinds thereof.

As the polymerization accelerator, tertiary amines can also be used in addition to the polyfunctional thiol compounds. Specific examples of the tertiary amines include N,N-dimethylaniline, N,N-diethylaniline, N,N-di-n-butylaniline, N,N-dibenzylaniline, N,N-dimethyl-p-toluidine, N,N-dimethyl-m-toluidine, N,N-diethyl-p-toluidine, p-bromo-N,N-dimethylaniline, p-m-chloro-N,N-dimethylaniline, dimethylaminobenzaldehyde, p-dimethylaminoacetophenone, p-dimethylaminobenzoic acid, p-dimethylaminobenzoic acid ethyl ester, p-dimethylaminobenzoic acid amino ester, N,N-dimethylanthranilic acid methyl ester, N,N-dihydroxyethylaniline, N,N-dihydroxyethyl-p-toluidine, p-dimethylaminophenyl alcohol, p-dimethylaminostyrene, N,N-dimethyl-3,5-xylidine, 4-dimethylaminopyridine, N,N-dimethyl-α-naphthylamine, N,N-dimethyl-β-naphthylamine, tributylamine, tripropylamine, triethylamine, N-methyldiethanolamine, N-ethyldiethanolamine, N,N-dimethylhexylamine, N,N-dimethyldodecylamine, N,N-dimethylstearylamine, N,N-dimethylaminoethyl methacrylate, N,N-diethylaminoethyl methacrylate, and 2,2′-(n-butylimino)diethanol. Here, the examples thereof are not limited thereto. These polymerization accelerators may be used singly or in combination of two or more kinds thereof.

The artificial nail composition of the present invention can be applied to a natural nail, a nail on which a coating is formed by the artificial nail composition, and a base material such as an artificial resin chip, a resin film and a resin sheet and is not limited thereto. In addition, examples of a method of application include methods of application using a brush, a sponge, a spray, an inkjet, an air knife, a roll, or the like and are not limited thereto.

The viscosity of the artificial nail composition of the present invention is not particularly limited, and the artificial nail composition preferably has fluidity under the 23° C. atmosphere from the viewpoint of operability. Specifically, a viscosity of the artificial nail composition of the present invention is preferably 50,000 mPa·s or less, more preferably 10,000 mPa·s or less, and still more preferably 5,000 mPa·s or less at 23° C. and a shear rate of 10 s⁻¹ as measured using a rheometer which is a dynamic viscoelasticity measuring device. On the other hand, a lower limit of the viscosity is not particularly limited and is preferably 1 mPa·s or more, more preferably 10 mPa·s or more, still more preferably 100 mPa·s or more, particularly preferably 1,000 mPa·s or more, and most preferably 1,500 mPa·s or more. Note that the viscosity of the artificial nail composition of the present invention can be adjusted by appropriately adjusting kinds and contents of the components (A) to (C).

Surface roughness of an artificial nail (cured product) obtained from the artificial nail composition of the present invention is not particularly limited, but, from the viewpoints of the mattness, the texture, the adhesion resistance, and the antifouling property, arithmetic average roughness (Ra) of a surface thereof is preferably within a range of 0.2 to 1.3 μm, more preferably within a range of 0.4 to 1.0 μm, and still more preferably within a range of 0.5 to 0.8 μm. Note that, in this specification, the arithmetic average roughness (Ra) of the surface of the artificial nail (cured product) is calculated by the following method. The artificial nail composition is applied onto plate glass by using a spacer and is cured by light irradiation for 20 seconds using a gel nail light (trade name: PRESTO LED Light manufactured by Nail Labo Corporation), then, a surface unpolymerized layer part of the artificial nail composition is removed using ethanol-soaked tissue paper, and a thin film having a thickness of 0.2 mm, a long side of 50.0 mm, and a short side of 15.0 mm is prepared. Note that a wiping step is omitted for a non-wipe type artificial nail composition of which removal of a surface unpolymerized layer does not need to be performed. Next, in accordance with “4.2.1” of JIS B 0601:2013 (Geometrical Product Specifications (GPS)—Surface texture: Profile method—Terms, definitions and surface texture parameters), surface roughness measurement of the prepared thin films was performed at random three points using a stylus type surface roughness measuring instrument (model: SURFCOM 1500SD manufactured by TOKYO SEIMITSU CO., LTD.), and an average value thereof was employed as the arithmetic average roughness (Ra).

The kind of artificial nail composition of the present invention is not particularly limited, and examples thereof include nail polish, a gel nail, and an acrylic nail. The nail polish is a coating material for a nail which is called a nail lacquer, a nail enamel, a manicure, or the like and is a composition for producing a coating excellent in aesthetic appearance by drying a contained solvent. The gel nail is a material containing a resin component that is cured by an ultraviolet ray or a visible light ray and is a composition that forms a coating excellent in aesthetic appearance by being applied to a natural nail or an artificial nail and then cured by irradiation with the ultraviolet ray or the visible light ray. The acrylic nail is a powder-liquid type material containing a polymer bead and polymerizable monomers and is a composition in which polymerization of the polymerizable monomers is initiated and cured by a peroxide contained in the polymer bead after powder and a liquid are mixed. The acrylic nail is characterized in that the acrylic nail can not only be applied but also be produced in a layered-up form, and is mainly used for extending a nail in many cases. Among the kinds of artificial nail composition, it is preferable that the artificial nail composition of the present invention is the gel nail. When the artificial nail composition of the present invention is formed into a gel nail, an artificial nail having mattness with a subdued gloss and a smooth texture can be formed.

In the gel nail, a base layer, a color layer, and a topcoat layer are generally laminated in this order on a natural nail or a base material, and a coating is formed. An application method of the artificial nail composition of the present invention is not particularly limited, and any one layer of the base layer, the color layer, and the topcoat layer can be formed. The artificial nail composition of the present invention is preferably used as a topcoat layer from the viewpoint of enabling the mattness with a subdued gloss and the smooth texture, which are the properties of the artificial nail composition of the present invention, to be most remarkably exhibited. In addition, the artificial nail composition of the present invention is also preferably used as a base layer that allows art to be performed, from the viewpoint of being able to impart the mattness with a subdued gloss, the smooth texture, the adhesion resistance which makes it difficult for mirror powder to adhere, and the transparency.

Examples of the gel nail include a gel nail for a hand which is applied to a nail of a hand, a gel nail for a foot which is applied to a nail of a foot, and a gel nail for an animal which is applied to a claw of an animal. The use of the artificial nail composition of the present invention is not particularly limited, and the artificial nail composition can be used for any gel nail for a hand, a foot, an animal, or the like.

### EXAMPLES

Hereinafter, Examples and Comparative Examples of the present invention will be specifically described, but the present invention is not limited to these Examples.

### [Components Used for Preparation of Artificial Nail Composition]

The components used for preparation of artificial nail compositions of Examples and Comparative Examples are described below. As the weight-average molecular weight (Mw), a value measured using tetrahydrofuran as an eluent and polystyrene as a standard substance from gel permeation chromatography (GPC) performed by a GPC measurement system (trade name: Nexera GPC System manufactured by Shimadzu Corporation) and a column (trade name: Styragel HR manufactured by Waters Corporation) is used. In addition, as the average particle size, a value of an integrated volume 50% particle size obtained by measuring a particle grain-size distribution with a laser diffraction particle grain-size distribution measuring machine was employed.

Abbreviated symbols shown in Tables 1 to 5 are as follows.
A1: urethane diacrylate oligomer (weight-average molecular weight: 6000)
A2: urethane diacrylate oligomer (weight-average molecular weight: 8400)
A3: urethane diacrylate oligomer (weight-average molecular weight: 1000)
A4: triethylene glycol dimethacrylate
A5: isobornyl methacrylate
A6: acryloylmorpholine
A7: tetrahydrofurfuryl methacrylate
A8: trimethylolpropane triacrylate
B1: cellulose particles (Viscopearl D-3, average particle size: 3 μm, manufactured by RENGO CO., LTD.)
B2: cellulose particles (CELLULOBEADS D-5, average particle size: 5 μm, manufactured by DAITO KASEI KOGYO CO., LTD.)
B3: cellulose acetate particles (BELLOCEA S7, average particle size: 7 μm, manufactured by Daicel Corporation)
B4: cellulose particles (CELLULOBEADS D-10, average particle size: 10 μm, manufactured by DAITO KASEI KOGYO CO., LTD.)
B5: cellulose particles (CELLULOBEADS D-30, average particle size: 30 μm, manufactured by DAITO KASEI KOGYO CO., LTD.)
B′1: acrylic polymer particles (Ganzpearl GMX-0810, average particle size: 8 μm, manufactured by Aica Kogyo Co., Ltd.)
C1: 2,4,6-trimethylbenzoyldiphenylphosphine oxide
C2: 2,4,6-trimethylbenzoylphenylethoxyphosphine oxide
C3: bis(2,4,6-trimethylbenzoyl)phenylphosphine oxide
R-SH: pentaerythritol tetrakis(3-mercaptopropionate)

### [Preparation of Artificial Nail Composition]

The individual components were calculated according to respective blending ratios shown in Tables 1 to 5 and mixed until a uniform liquid is produced under atmospheric pressure using a rotation/revolution type mixer (trade name: ARV-310P manufactured by THINKY CORPORATION), thereby preparing artificial nail compositions of Examples and Comparative Examples.

An evaluation method of the artificial nail compositions of Examples and Comparative Examples is as follows. Note that all evaluations were performed under indoor LED lighting at a room temperature of 23±2° C. and a humidity of 50±10%.

### Test Example 1: Mattness Evaluation

The artificial nail compositions of Examples and Comparative Examples were applied onto respective pieces of plate glass by using a spacer and were cured by light irradiation for 20 seconds using the gel nail light (trade name: PRESTO LED Light manufactured by Nail Labo Corporation), and then a surface unpolymerized layer part of the artificial nail composition was removed using ethanol-soaked tissue paper, and thin films having a thickness of 0.2 mm, a long side of 50.0 mm, and a short side of 15.0 mm were prepared. Note that the wiping step was omitted for Example 18 of a non-wipe type of which removal of a surface unpolymerized layer does not need to be performed. Next, gloss at 60° was measured using a gloss meter (model: HORIBA Gloss Checker IG-331 manufactured by HORIBA, Ltd.). In addition, three thin films were prepared for one artificial nail composition, and the mattness was evaluated. Note that a mattness evaluation value is an average value of gloss levels of the three thin films. When the mattness evaluation value is 30.0 or less, it is determined that the thin films have the mattness, and the mattness evaluation value is preferably 10.0 or less, and more preferably 5.0 or less.

### Test Example 2: Transparency Evaluation

The artificial nail compositions of Examples and Comparative Examples were applied onto respective pieces of plate glass by using a spacer and were cured by light irradiation for 20 seconds using the gel nail light (trade name: PRESTO LED Light manufactured by Nail Labo Corporation), and then a surface unpolymerized layer part of the artificial nail composition was removed using ethanol-soaked tissue paper, and thin films having a thickness of 0.2 mm, a long side of 50.0 mm, and a short side of 15.0 mm were prepared. Note that the wiping step was omitted for Example 18 of a non-wipe type of which removal of a surface unpolymerized layer does not need to be performed. Next, ten manicurists certified by the Japan Nailist Association visually performed sensory evaluations on the transparency of the thin films, and evaluation results according to the following criteria were brought together, and a comprehensive evaluation was performed. When an evaluation result is A or B from the comprehensive evaluation, the transparency can be said to be good.

### <Evaluation Criteria>

A: Acceptably cloudy
B: Slightly cloudy
C: Cloudy and opaque
D: Not evaluated because of no mattness

### Test Example 3: Texture Test

The artificial nail compositions of Examples and Comparative Examples were applied onto respective pieces of plate glass by using a spacer and were cured by light irradiation for 20 seconds using the gel nail light (trade name: PRESTO LED Light manufactured by Nail Labo Corporation), and then a surface unpolymerized layer part of the artificial nail composition was removed using ethanol-soaked tissue paper, and thin films having a thickness of 0.2 mm, a long side of 50.0 mm, and a short side of 15.0 mm were prepared. Note that the wiping step was omitted for Example 18 of a non-wipe type of which removal of a surface unpolymerized layer does not need to be performed. Next, ten manicurists certified by the Japan Nailist Association touched a light-irradiated surfaces of the thin films with their fingertips cleaned with ethanol, sensory evaluation was performed on the textures of the thin films, and evaluation results according to the following criteria were brought together, and a comprehensive evaluation was performed. In a case where an evaluation result is A or B from the comprehensive evaluation, the texture can be said to be good.

### <Evaluation Criteria>

A: With a soft and extremely smooth texture
B: With a soft and smooth texture
C: With a rough texture lacking smoothness
D: With a rough powdery texture
E: Not evaluated because of no mattness

### Test Example 4: Surface Roughness Measurement

The artificial nail compositions of Examples and Comparative Examples were applied onto respective pieces of plate glass by using a spacer and were cured by light irradiation for 20 seconds using the gel nail light (trade name: PRESTO LED Light manufactured by Nail Labo Corporation), and then a surface unpolymerized layer part of the artificial nail composition was removed using ethanol-soaked tissue paper, and thin films having a thickness of 0.2 mm, a long side of 50.0 mm, and a short side of 15.0 mm were prepared. Note that the wiping step was omitted for Example 18 of a non-wipe type of which removal of a surface unpolymerized layer does not need to be performed. Next, in accordance with “4.2.1” of JIS B 0601:2013 (Geometrical Product Specifications (GPS)—Surface texture: Profile method—Terms, definitions and surface texture parameters), surface roughness measurement of the prepared thin films was performed at random three points using a stylus type surface roughness measuring instrument (model: SURFCOM 1500SD manufactured by TOKYO SEIMITSU CO., LTD.), and an average value thereof was employed as the arithmetic average roughness (Ra).

### Test Example 5: Adhesion Resistance of Mirror Powder

The artificial nail compositions of Examples and Comparative Examples were applied onto respective pieces of plate glass by using a spacer and were cured by light irradiation for 20 seconds using the gel nail light (trade name: PRESTO LED Light manufactured by Nail Labo Corporation), and then a surface unpolymerized layer part of the artificial nail composition was removed using ethanol-soaked tissue paper, and thin films having a thickness of 0.2 mm, a long side of 15.0 mm, and a short side of 15.0 mm were prepared. Note that the wiping step was omitted for Example 18 of a non-wipe type of which removal of a surface unpolymerized layer does not need to be performed. Next, ten manicurists certified by the Japan Nailist Association uniformly sprinkled 5 mg of commercially available mirror powder (Ageha Mirror Powder Gold M-2) on the light-irradiated surfaces of the thin films, then brushed the mirror powder using a commercially available nail duster brush (PREANFA Dust Brush), and visually performed sensory evaluations on the adhesion of the mirror powder, and evaluation results according to the following criteria were brought together, and a comprehensive evaluation was performed. Note that, from the viewpoint of using the artificial nail composition as a base layer of art, an evaluation result from the comprehensive evaluation is preferably A or B, and more preferably A.

### <Evaluation Criteria>

A: The mirror powder was removed by performing brushing ten times or less.
B: The mirror powder was removed by performing brushing eleven times to thirty times.
C: The mirror powder remained even after brushing was performed thirty times or more.

### Test Example 6: Viscosity Measurement

Regarding the artificial nail compositions of Examples and Comparative Examples, the viscosity measurement was performed under the following measurement conditions using the rheometer (model: Physica MCR 301 manufactured by Anton Paar GmbH) which is the dynamic viscoelasticity measuring device, and a measured value at a shear rate of 10 s⁻¹ was set as a viscosity value.

### (Measurement Conditions)

Measurement jig: Parallel plate PP20
Shear rate: 0.1 to 100 s⁻¹
Measurement temperature: 23° C.

### (Evaluation Results of Examples and Comparative Examples)

Tables 1 to 5 show the measurement and the evaluation results of the mattness, the transparency, the texture, the arithmetic average roughness, the adhesion resistance of the mirror powder, and the viscosity of Examples and Comparative Examples.

TABLE 1

| | Example 1 | Example 2 | Example 3 | Example 4 | Example 5 | Example 6 |
|---|---|---|---|---|---|---|
| A1 | 32.4 | 30.7 | 27.3 | 22.2 | 17.1 | 13.7 |
| A4 | 31.4 | 29.8 | 26.5 | 21.5 | 16.5 | 13.2 |
| A5 | 29.8 | 28.2 | 25.0 | 20.3 | 15.6 | 12.5 |
| B2 | 5.0 | 10.0 | 20.0 | 35.0 | 50.0 | 60.0 |
| C1 | 1.4 | 1.3 | 1.2 | 1.0 | 0.8 | 0.6 |
| Total | 100.0 | 100.0 | 100.0 | 100.0 | 100.0 | 100.0 |
| Mattness | 27.0 | 8.7 | 4.0 | 3.3 | 1.0 | 1.0 |
| evaluation | | | | | | |
| Transparency | A | A | A | A | B | B |
| evaluation | | | | | | |
| Texture test | B | A | A | A | A | B |
| Surface | 0.2 | 0.6 | 0.8 | 0.7 | 1.0 | 1.3 |
| roughness | | | | | | |
| measurement | | | | | | |
| Ra/μm | | | | | | |
| Adhesion | B | B | A | A | A | A |
| resistance of | | | | | | |
| mirror powder | | | | | | |
| Viscosity | 1.5 | 1.6 | 1.8 | 1.8 | 1.9 | 2.0 |
| measurement/ | | | | | | |
| Pa · s | | | | | | |

TABLE 2

| | Example 7 | Example 8 | Example 9 | Example 10 |
|---|---|---|---|---|
| A1 | 22.2 | 22.2 | 22.2 | 22.2 |
| A4 | 21.5 | 21.5 | 21.5 | 21.5 |
| A5 | 20.3 | 20.3 | 20.3 | 20.3 |
| B1 | 35.0 | | | |
| B3 | | 35.0 | | |
| B4 | | | 35.0 | |
| B5 | 1.0 | 1.0 | 1.0 | 35.0 |
| C1 | | | | 1.0 |
| Total | 100.0 | 100.0 | 100.0 | 100.0 |
| Mattness evaluation | 5.3 | 3.7 | 2.7 | 2.7 |
| Transparency | A | A | B | A |
| evaluation | | | | |
| Texture test | A | A | B | A |
| Surface roughness | 0.7 | 0.8 | 1.1 | 0.5 |
| measurement Ra/μm | | | | |
| Adhesion resistance | A | A | B | A |
| of mirror powder | | | | |
| Viscosity | 1.8 | 1.9 | 2.3 | 2.0 |
| measurement/Pa · s | | | | |

TABLE 3

| | Example 11 | Example 12 | Example 13 | Example 14 | Example 15 |
|---|---|---|---|---|---|
| A1 | 22.2 | 22.2 | 22.2 | 22.2 | 22.2 |
| A4 | 21.5 | 21.5 | 21.5 | 21.5 | 21.5 |
| A5 | 20.3 | 20.3 | 20.3 | 20.3 | 20.3 |
| B1 | 10.0 | | | | |
| B2 | 25.0 | 17.5 | 17.5 | | 28.0 |
| B3 | | 17.5 | | 17.5 | |
| B4 | | | | | 7.0 |
| B5 | | | 17.5 | 17.5 | |
| C1 | 1.0 | 1.0 | 1.0 | 1.0 | 1.0 |
| Total | 100.0 | 100.0 | 100.0 | 100.0 | 100.0 |
| Mattness evaluation | 4.7 | 3.3 | 3.3 | 3.0 | 2.8 |
| Transparency | A | A | A | A | B |
| evaluation | | | | | |
| Texture test | A | A | A | A | B |
| Surface roughness | 0.7 | 0.8 | 0.7 | 0.8 | 1.0 |
| measurement Ra/μm | | | | | |
| Adhesion resistance | A | A | A | A | A |
| of mirror powder | 2.1 | 1.9 | 1.8 | 1.9 | 2.2 |
| Viscosity | | | | | |

TABLE 4

| | Example 16 | Example 17 | Example 18 | Example 19 | Example 20 |
|---|---|---|---|---|---|
| A1 | 26.0 | | | 22.2 | 22.2 |
| A2 | | 29.0 | | | |
| A3 | | | 17.4 | | |
| A4 | 15.2 | 24.3 | | 21.5 | 21.5 |
| A5 | | 10.5 | | 20.3 | 20.3 |
| A6 | 18.6 | | 29.6 | | |
| A7 | 4.0 | | | | |
| A8 | | | 7.0 | | |
| B2 | 35.0 | 35.0 | 35.0 | 35.0 | 35.0 |
| C1 | 1.2 | 1.2 | 4.0 | | |
| C2 | | | | 1.0 | |
| C3 | | | | | 1.0 |
| R-SH | | | 7.0 | | |
| Total | 100.0 | 100.0 | 100.0 | 100.0 | 100.0 |
| Mattness evaluation | 4.0 | 4.3 | 5.7 | 3.7 | 4.0 |
| Transparency | A | A | A | A | A |
| evaluation | | | | | |
| Texture test | A | A | A | A | A |
| Surface roughness | 0.7 | 0.6 | 0.4 | 0.7 | 0.8 |
| measurement Ra/μm | | | | | |
| Adhesion resistance | A | A | A | A | A |
| of mirror powder | | | | | |
| Viscosity | 2.1 | 4.8 | 3.7 | 1.8 | 1.8 |
| measurement/Pa · s | | | | | |

TABLE 5

| | Comparative | Comparative | Comparative | Comparative |
|---|---|---|---|---|
| | Example 1 | Example 2 | Example 3 | Example 4 |
| A1 | 22.2 | 27.3 | 22.2 | 17.1 |
| A4 | 21.5 | 26.5 | 21.5 | 16.5 |
| A5 | 20.3 | 25.0 | 20.3 | 15.6 |
| B'1 | | 20.0 | 35.0 | 50.0 |
| C1 | 1.0 | 1.2 | 1.0 | 0.8 |
| Total | 65.0 | 100.0 | 100.0 | 100.0 |
| Mattness | 91.0 | 6.0 | 3.0 | 1.3 |
| evaluation | | | | |
| Transparency | D | A | B | C |
| evaluation | | | | |
| Texture test | E | C | C | D |
| Surface | 0.1 | 1.1 | 1.2 | 1.4 |
| roughness | | | | |
| measurement | | | | |
| Ra/μm | | | | |
| Adhesion | C | C | C | C |
| resistance | | | | |
| of mirror | | | | |
| powder | | | | |
| Viscosity | 1.5 | 2.5 | 6.8 | 17.0 |
| measurement/ | | | | |
| Pa · s | | | | |

As is clarified from Tables 1 to 5, each of the artificial nail compositions according to Examples had the mattness and the smooth texture and further had good transparency, the adhesion resistance of the mirror powder, and good operability. On the other hand, each of the artificial nail compositions of Comparative Examples could not achieve both the mattness and the smooth texture.

According to the present invention, it is possible to provide the artificial nail composition having the mattness with a subdued gloss and the smooth texture.

1. An artificial nail composition comprising:
a component (A): a polymerizable compound;
a component (B): cellulose particles; and
a component (C): a polymerization initiator.

2. The artificial nail composition according to claim 1, wherein the component (B) that is cellulose particles has an average particle size of 3 to 30 μm.

3. The artificial nail composition according to claim 1, wherein a content of the component (B) that is cellulose particles is 5 to 60 mass % with respect to a total amount of the artificial nail composition.

4. The artificial nail composition according to claim 1, wherein the component (A) that is a polymerizable compound is at least one selected from the group consisting of a urethane (meth)acrylate oligomer and a polymerizable monomer.

5. The artificial nail composition according to claim 4, wherein the urethane (meth)acrylate oligomer is at least one selected from the group consisting of a urethane mono(meth)acrylate oligomer, a urethane di(meth)acrylate oligomer, and a urethane hexa(meth)acrylate oligomer.

6. The artificial nail composition according to claim 4, wherein the polymerizable monomer is at least one selected from the group consisting of isobornyl (meth)acrylate, tetrahydrofurfuryl (meth)acrylate, hydroxypropyl (meth)acrylate, triethylene glycol di(meth)acrylate, (meth)acryloylmorpholine, trimethylolpropane tri(meth)acrylate, ethoxyethylene glycol (meth)acrylate, and isoprenyl (meth)acrylate.

7. The artificial nail composition according to claim 1, wherein the component (C) that is a polymerization initiator is a photopolymerization initiator.

8. The artificial nail composition according to claim 1, comprising:
39 to 94 mass % of the component (A): a polymerizable compound,
5 to 60 mass % of the component (B): cellulose particles; and
0.1 to 10 mass % of the component (C): polymerization initiator.

9. An artificial nail obtained from the artificial nail composition according to claim 1.

10. The artificial nail according to claim 9, wherein a surface of the artificial nail has arithmetic average roughness (Ra) of 0.2 to 1.3 μm.

11. An artificial nail obtained from the artificial nail composition according to claim 2.

12. An artificial nail obtained from the artificial nail composition according to claim 3.

13. An artificial nail obtained from the artificial nail composition according to claim 4.

14. An artificial nail obtained from the artificial nail composition according to claim 5.

15. An artificial nail obtained from the artificial nail composition according to claim 6.

16. An artificial nail obtained from the artificial nail composition according to claim 7.

17. An artificial nail obtained from the artificial nail composition according to claim 8.
